(19) United States
(12) Patent Application Publication
(10) Pub. No.: US 2004/0029146 A1
(43) Pub. Date: Feb. 12, 2004

(54) Polymorphic repetitive sequences in chlamydiae and uses thereof

(76) Inventors: Erick Denamur, Paris (FR); Eduardo Rocha, Paris (FR); Chalom B. Sayada, Luxembourg City (FR)

Correspondence Address: CLARK & ELBING LLP, 101 FEDERAL STREET, BOSTON, MA, 02110, US

(21) Appl. No.: 10/393,409

(22) Filed: Mar. 20, 2003

Related U.S. Application Data
(60) Provisional application No. 60/366,477, filed on Mar. 21, 2002.

(51) Int. Cl. C12Q 1/68

(52) U.S. Cl. 435/6

(57) ABSTRACT

In general, the invention features a method for determining the presence of a strain of chlamydia in a biological sample. The method includes the steps of (a) providing a biological sample; and (b) determining the presence of a polynucleotide containing a polymorphic repetitive sequence in a polynucleotide in the sample, wherein the polymorphic repetitive sequence is associated with a first strain of chlamydia and not associated with a second strain of chlamydiae. In this method, the presence of the polynucleotide containing the polymorphic repetitive sequence indicates presence of the first strain of chlamydia.

### CROSS-REFERENCE TO RELATED APPLICATIONS

This application claims the benefit of U.S. Provisional Application No. 60/366,477, filed Mar. 21, 2002.

### BACKGROUND OF THE INVENTION

The invention relates to the field of diagnosis and treatment of infectious diseases.

The chlamydiae are obligate intracellular pathogens that cause a variety of diseases in animal species at virtually every phylogenetic level. Of these, Chlamydia (C.) trachomatis and C. pneumoniae are considered the most significant human pathogens. C. trachomatis is the leading cause of preventable blindness worldwide and the most common sexually transmitted bacterial species. C. pneumoniae causes 10% to 20% of community-acquired pneumonia worldwide and has recently been associated with coronary arteriosclerosis and multiple sclerosis. The chlamydiae undergo a developmental cycle unique among prokaryotes. The elementary body is infectious, but is metabolically inactive and cannot replicate. This form differentiates upon infection into the non-infectious reticulate body, a larger pleomorphic bacterium that is metabolically active and multiplies. Following uptake, chlamydiae develop and grow within an intracellular vacuole, called an inclusion, where they will differentiate from the elementary body to the reticulate body then to the elementary body.

Chlamydiae encode an abundant protein termed the major outer membrane protein (MOMP, or OmpA) that is surface exposed in C. psittaci and C. trachomatis and is the major determinant for serologic classification of chlamydial isolates. This protein is highly variable within its exposed domains except in ruminant invasive C. psittaci, feline strains of C. psittaci and C. pneumoniae, where they are extremely conserved.

Completion of the sequences of five chlamydial genomes (one C. trachomatis, three C. pneumoniae and one C. muridarum) has revealed the importance of a group of proteins unique to the chlamydiae, the polymorphic membrane proteins (Pmps). These proteins had been shown previously to be antigenic in C. psittaci. The genes encoding for these proteins belong to a complex family and span 13.6 and 17.5% of the C. trachomatis and C. pneumoniae genomes, respectively. There is a considerable expansion of these genes in C. pneumoniae; the C. trachomatis genome possesses 9 pmp genes (A to I) whereas the C. pneumoniae genome possesses 21 pmp genes. Pmps are characterized by two repeated tetrapeptidic motifs, almost never found outside chlamydiae: GGA(L/V/I) (SEQ ID NO: 1) and FXXN (SEQ ID NO: 2). The non-chlamydial proteins exhibiting these motifs have been implicated in the adherence to mammalian tissues. As the Pmps have been localized at the chlamydial cell surface, their role in adhesion, molecular transport, signaling, or some other cell wall associated function is likely.

Prokaryotic genomes are compact, with sizes ranging from less than 600 kb in Mycoplasma to more than 10 Mb in several cyanobacterial and myxobacterial species. Chlamydial genomes range from 1 to 1.2 Mb. These compact genomes have likely been maintained through selective pressure for rapid DNA replication and cell reproduction. Furthermore, the obligate intracellular way of life of the chlamydiae tends to minimize the length of the genome. It was therefore expected that repetitive sequences would be kept to a minimum under natural selection for rapid growth. Various classes of repetitive DNA elements have been recently discovered in many prokaryotes (Rocha et al., Mol. Biol. Evol. 16:1219-1230, 1999). Such repetitive sequences can be the cause or the hallmark of the plasticity of the genome. Thus, bacteria could have evolved mechanisms based on the presence of repeated sequences for increasing the frequency of random variations in a specific subset of genes. Molecular mechanisms at the basis of this variation are essentially based on slipped-mispair of replicating strands for close repeats and homologous recombination between long intra-chromosomal repeats. These highly mutable loci, sometimes called ‘contingency’ loci, would be involved in critical interactions with the environment, allowing certain phenotypic traits to respond rapidly, by natural selection, to unpredictable changes.

### SUMMARY OF THE INVENTION

Using an in silico approach, we have examined repeats within the complete genomes of chlamydiae. This analysis focused on the search for repeats of statistically significant length, taking into account the genome size and composition. We then determined whether those repeats were sites for sequence variation in vivo.

We discovered that the repeated sequences in different strains of chlamydiae were polymorphic. The presence of a particular polymorphism can thus be used to detect the presence of a particular strain by detecting the presence of a polymorphic repeated sequence associated with that strain and not associated with other strains of chlamydiae.

Accordingly, in a first aspect, the invention features a method for determining the presence of a strain of chlamydia in a biological sample. The method includes the steps of (a) providing a biological sample; and (b) determining the presence of a polynucleotide containing a polymorphic repetitive sequence in a polynucleotide in the sample, wherein the polymorphic repetitive sequence is associated with one strain of chlamydia and not associated with other strains of chlamydiae. In this method, the presence of the polynucleotide containing the polymorphic repetitive sequence indicates the presence of that strain of chlamydia.

In a second, related aspect, the invention features a method for determining the presence of a plurality of strains of chlamydiae in a biological sample. This method includes the steps of: (a) providing a biological sample; and (b) determining the presence in the biological sample of a plurality of polynucleotides, each containing a polymorphic repetitive sequence, wherein each polymorphic repetitive sequence is associated with one strain of chlamydia and not associated with other strains of chlamydiae. In this method, presence of a polymorphic repetitive sequence indicates the presence of the strain of chlamydia associated with that polymorphic repetitive sequence, and the absence of that polymorphic repetitive sequence indicates absence of the associated strain of chlamydia.

In another aspect, the invention features a method for treating a chlamydial infection in a patient. This method includes the steps of (a) providing a biological sample from the patient; (b) determining the presence in the biological sample of a plurality of polynucleotides, each containing a polymorphic repetitive sequence, wherein each polymorphic repetitive sequence is associated with one strain of chlamydia and not associated with other strains of chlamydiae; and (c) administering to the patient anti-chlamydial agents that are effective against the strains of chlamydiae that are present in the biological sample.

In any of the foregoing methods, the strain of chlamydia can be a strain of any chlamydial species (e.g., C. psittaci, C. trachomatis, C. pecorum, C. abortus, C. caviae, C. felis, C. suis, C. muridarum, Neochlamydia (N.) hartmannellae, Parachlamydia (P.) acanthamoebae, Simkania (S.) negevensis, and Waddlia (W.) chondrophila). In particular embodiments, the strain of chlamydia is C. pneumoniae strain CWL-029, C. pneumoniae strain AR 39, C. pneumoniae strain J138, or C. trachomatis strain D/UW-3/Cx.

The polymorphic repetitive sequence can be a simple sequence repeat (SSR); a small close or tandem repeat (TR); or a large repeat (LR). Exemplary SSRs and their locations are listed in Tables 1, 5, 9, 13, and 16, below. The locations of exemplary TRs and LRs are listed in Tables 2-4, 6-8, 10-12, 14, 15, and 17-19.

The biological sample can be a biopsy sample, blood, serum, peripheral blood mononuclear cells, cerebrospinal fluid, urine, nasal secretion, saliva, or any other biological sample that may contain chlamydiae. The method of detecting the presence of a polymorphic repetitive sequence can include any suitable polynucleotide detection step, e.g., by amplification of polynucleotide molecules that contain a polymorphic repetitive sequence.

By “chlamydia” or “chlamydiae” is meant organisms of the order Chlamydiales. Examples include, but are not limited to, C. psittaci, C. trachomatis, C. pecorum, C. abortus, C. caviae, C. felis, C. suis, C. muridarum, N. hartmannellae, P. acanthamoebae, S. negevensis, and W. chondrophila. By “chlamydial infection” is meant an infection of a cell or organism by an organism of the order Chlamydiales.

By “polypeptide” is meant any chain of more than two amino acids, regardless of post-translational modification such as glycosylation or phosphorylation.

In another aspect, the invention features a purified polypeptide that is substantially identical to a POMP2 polypeptide of SEQ ID NO: 3, SEQ ID NO: 4, or SEQ ID NO: 5, or a POMP4 polypeptide of SEQ ID NO: 6, SEQ ID NO: 7, or SEQ ID NO: 8.

In a related aspect, the invention features a purified polynucleotide encoding a polypeptide that is substantially identical to a POMP2 polypeptide of SEQ ID NO: 3, SEQ ID NO: 4, or SEQ ID NO: 5, or a POMP4 polypeptide of SEQ ID NO: 6, SEQ ID NO: 7, or SEQ ID NO: 8.

In still another aspect, the invention features a method of identifying a compound useful for treating or preventing an infection of C. pneumoniae. This method includes the steps of: (a) contacting a candidate compound and a POMP polypeptide; and (b) determining the specific binding of the candidate compound to the POMP polypeptide. A candidate compound that specifically binds to the POMP polypeptide is identified as a compound useful for treating or preventing an infection of C. pneumoniae.

The invention features another method of identifying a compound useful for treating or preventing an infection of C. pneumoniae. This method includes the steps of: (a) contacting a candidate compound and a POMP polynucleotide; and (b) determining the specific binding of the candidate compound to the polynucleotide, wherein a candidate compound that specifically binds to the polynucleotide is identified as a compound useful for treating or preventing an infection of C. pneumoniae.

The invention also features a method of immunizing a subject against an infection of C. pneumoniae by administering to the subject a purified POMP polypeptide or an immunogenic fragment thereof in an amount sufficient to induce an immune response to the POMP polypeptide or fragment thereof.

In still other aspects, the invention features a peptide fragment of a POMP2 or POMP4polypeptide, an isolated antibody that specifically binds a POMP2 or POMP4 polypeptide, an antigenic composition that includes a POMP2 or POMP4 polypeptide (or a fragment thereof) and a pharmaceutically acceptable carrier or diluent, and a pharmaceutical composition that includes an antibody that specifically binds a POMP2 or POMP4 polypeptide and a pharmaceutically acceptable carrier or diluent.

The invention also features a method of producing an immune response in an animal by immunizing the animal with an effective amount of a POMP polypeptide (e.g., a POMP2 or POMP4 polypeptide) or a peptide fragment of a POMP polypeptide.

POMP polypeptides that are a part of the invention include those that are substantially identical to C. pneumoniae POMP2 or POMP4 (FIGS. 2A-2C and 3A-3C, respectively). POMP polynucleotides that are a part of the invention include those encoding POMP polypeptides as defined above, as well as polynucleotides substantially identical to POMP1, POMP2, POMP3, POMP4, POMP5, POMP6, or POMP7 (FIG. 1).

By “substantially identical” is meant a polypeptide or polynucleotide exhibiting at least 95%, 99%, 99.5%, or 99.9%, identity to a reference amino acid or polynucleotide sequence. For polypeptides, the length of comparison sequences will generally be at least 16 amino acids, preferably at least 20 amino acids, more preferably at least 25 amino acids, and most preferably 35 amino acids. For polynucleotides, the length of comparison sequences will generally be at least 50 nucleotides, preferably at least 60 nucleotides, more preferably at least 75 nucleotides, and most preferably 110 nucleotides.

Sequence identity is typically measured using sequence analysis software with the default parameters specified therein (e.g., BLAST 2 (Tatusova et al., FEMS Microbiol Lett. 174:247-250, 1999); Sequence Analysis Software Package of the Genetics Computer Group, University of Wisconsin Biotechnology Center, 1710 University Avenue, Madison, Wis. 53705). These programs match similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

By “high stringency conditions” is meant hybridization in 2+ SSC at 40° C. with a DNA probe length of at least 40 nucleotides. For other definitions of high stringency conditions, see F. Ausubel et al., Current Protocols in Molecular Biology, pp. 6.3.1-6.3.6, John Wiley & Sons, New York, N.Y., 1994, hereby incorporated by reference.

### DESCRIPTION OF THE DRAWINGS

FIG. 1 is a schematic illustration showing a family of POMP elements, their positions in three strains of C. pneumoniae, the number of cytidines in the SSR, and the genes annotated for their region.

FIG. 2A is a schematic illustration showing the amino acid sequence of POMP2 from C. pneumoniae strain CWL-029.

FIG. 2B is a schematic illustration showing the amino acid sequence of POMP2 from C. pneumoniae strain J138.

FIG. 2C is a schematic illustration showing the amino acid sequence of POMP2 from C. pneumoniae strain AR 39 .

FIG. 3A is a schematic illustration showing the amino acid sequence of POMP4 from C. pneumoniae strain CWL-029 .

FIG. 3B is a schematic illustration showing the amino acid sequence of POMP4 from C. pneumoniae strain J138.

FIG. 3C is a schematic illustration showing the amino acid sequence of POMP4 from C. pneumoniae strain AR 39.

Other features and advantages of the invention will be apparent from the following description of the preferred embodiments thereof.

### DETAILED DESCRIPTION OF THE INVENTION

Using algorithms designed to search for different types of repeats, we identified three classes of statistically significant repeats in the complete genomes of sequenced chlamydiae species (C. pneumoniae CWL-029, AR 39, J138, C. trachomatis D/UW-3/Cx, C. muridarum). These include (1) simple sequence repeats (SSRs); (2) small close or tandem repeats (TRs); and (3) large repeats (LRs). TRs and SSRs are thought to change by slipped-mispair at the time of replication or by single-strand annealing when the sequence faces double-strand breaks. Both mechanisms can result in conversion or deletion, but slipped mispair may also result in multiplication. LRs are thought to vary by homologous recombination, and this can lead to conversion or deletion. Additionally, recombination between direct LRs can result in multiplication, whereas recombination between inverted LRs may result in inversion. Hence, different repeats represent different recombination potentials that may result in substantially different outputs.

We have found when looking at a large collection of strains that these repeated sequences are polymorphic. Thus, the pmp_{—}10.2 cytosine stretch has been shown to be variable within C. pneumoniae strains, resulting in a shift out of frame in CWL-029 but not in AR 39 or TW- 183 (Grimwood et al., Infect. Immun. 69:2383-2389, 2001). Another difference between C. pneumoniae strains is a 393 nucleotide sequence (coding for 131 amino acids) in the 5′ part of pmp_{—}6, which is present three times in CWL-029 and J138 but only two times in AR 39.

These polymorphisms can be used as molecular markers that might differentiate strains bearing a conserved MOMP. The identification of subgroups within these groups (ruminant invasive C. psittaci, feline C. psittaci, and C. pneumoniae) should allow the search for correlations with the virulence and the different observed clinical syndromes.

Polymorphisms within simple and tandem repeats, according to their position in a coding or non-coding region, will generate a stop codon (if the modification in length is not a multiple of three) or a modification of the length of the promoter. Both mechanisms will lead to a modulation of the functional protein. The presence of polymorphisms allows for the identification of particular strains based on the presence of a particular polymorphism or pattern of polymorphisms.

Diagnostic Assays

As the presence of a particular polymorphic repetitive sequence is likely to correlate with the presence of a particular strain of chlamydiae, the invention features a method for determining the presence of one or more strains of chlamydiae in a patient. In the methods of the invention, a sample from an individual, such as an individual who is suspected of having a chlamydial infection or a disease associated with a chlamydial infection, is used. The test sample can include blood, serum, cerebrospinal fluid, urine, nasal secretion, saliva, or any other bodily fluid or tissue, or polynucleotides isolated from one of the foregoing samples.

The sample can be assayed for the presence or absence of the polymorphic repetitive sequence by Southern hybridization using a detectable probe for the appropriate polymorphic repetitive sequence. Alternatively, the test sample can be assayed using quantitative PCR or RT-PCR (e.g., by using a LightCycler™ (Idaho Technology Inc., Idaho Falls, Id.) and fluorescent LightCycler™ probes). The presence of the polymorphic repetitive sequence in the test sample is indicative of the presence of chlamydiae in the test sample. To facilitate assaying a test sample for the presence or absence of chlamydiae by detecting the presence or absence of a polymorphic repetitive sequence, the test sample can be subjected to methods to enhance isolation of chlamydia elementary bodies from the test sample and to release DNA from the elementary bodies. For example, elementary bodies have a tendency to adhere to the walls of a receptacle containing them; the elementary bodies can be removed from the receptacle by treating the receptacle containing the elementary bodies with trypsin/EDTA, thereby releasing elementary bodies that adhered to the receptacle; and then concentrating the released elementary bodies, such as by centrifugation or filtration. To release DNA from elementary bodies, the elementary bodies are incubated under disulfide reducing conditions, such as incubating the elementary bodies with a disulfide reducing agent such as dithiothreitol (DTT) or 2-mercaptoethanol; and digesting the elementary bodies with a protease (see, e.g., U.S. Pat. No. 6,258,532, hereby incorporated by reference).

The test sample can also be assayed for the presence of chlamydiae by detecting the presence of a polymorphic repetitive sequence in a protein from chlamydia. For example, the presence of a PMP protein having a particular polymorphic repetitive sequence in the test sample can be detected through the use of ELISA methodologies with an antibody that specifically recognizes the polymorphic repetitive sequence. Alternatively, the test sample may be assayed for the presence of chlamydiae by detecting the presence of human antibodies to polymorphic repetitive sequences in the test sample. The presence of a polymorphic repetitive sequence or antibodies to a polymorphic repetitive sequence in the test sample is indicative of the presence of chlamydiae in the test sample. The presence of proteins or antibodies may be detected by appropriate methods such as by ELISA, western blot, or isoelectric focusing.

The diagnostic methods described herein are useful for detecting or confirming the disease in a patient, as well as for monitoring the progress of the disease. Disease monitoring is useful, for example, for determining the efficacy of a particular therapy.

The invention will be further illustrated by the following non-limiting examples.

Identification of Polymorphic Repetitive Sequences

The tables containing the elements found in the five chlamydia genomes follow below ordered by genome and by repeat type. The GenBank accession identification numbers are as follows: AE001363 (C. pneumoniae CWL-029; Kalman et al., Nature Genet. 21:385-389, 1999); AE002161 (C. pneumoniae AR 39; Read et al., Nucleic Acids Res. 28:1397-1406, 2000); BA000008 (C. pneumoniae J138; Shirai et al., Nucleic Acids Res. 28:2311-2314, 2000); AE001273 (C. trachomatis; Stephens et al., Science 282:754-759, 1998); AE002160 (C. muridarum; Read et al., Nucleic Acids Res. 28:1397-1406, 2000). “ID” indicates an identification tag, “position” indicates the position of the start of the repeat in the respective genomes, “length” indicates the repeat length, “gene” indicates the gene where the repeat was found (if applicable), “sense” indicates if the repeat in the direct (d) or inverse (i) strand, “equivalent to” indicates the equivalent elements of the other genomes, “note” includes either the strand of the gene where the repeat stands or the flanking genes, in which case “D/C” stands for the position of the genes (direct or complement strands), and UFO indicates an unknown function ORF. For large repeats, “first” refers to the first occurrence of the repeat and “second” to the second occurrence. “Period” is in the form A×B, wherein “A” indicates the number of times the motif is repeated and “B” indicates the length of repeat. By “-” is meant that a consensus cannot be established to determine A and B with precision.

TABLE 1
Chlamydia pneumoniae strain CWL-029 SSRs

| ID | Position | length | gene | sense | note | equivalent to |
|---|---|---|---|---|---|---|
| | C(G)_{N}(N>11) | | | | | |
| C1 | 10806 | 14 | INT | d | D/UFO/CPn0007 D/UFO/CPn0008 | J1, A4 |
| C2 | 13350 | 14 | INT | d | D/UFO/CPn0009 D/UFO/CPn0010 | J2 |
| C3 | 20588 | 14 | pmp_2 | d | D | A3, J3 |
| C4 | 58474 | 14 | CPn0043 | d | D | A2, J4 |
| C5 | 85336 | 14 | CPn0069 | d | D | — |
| C6 | 507200 | 13 | pmp_10.2 | d | C | J5, A1 |
| C7 | 1207061 | 13 | CPn1054 | d | D | J6 |
| C8 | 1209609 | 12 | INT | d | D/UFO/CPn1054 D/UFO/CPn1055 | A5, J7 |
| | ACC_{N} /CAC_{N}(N>3) | | | | | |
| C9 | 628400 | 14 | CPn0542 | d | D/ABC transporter | J8, A9 |
| | TCC_{N} (N>4) | | | | | |
| C10 | 1150530 | 15 | ftsH | d | D/protease | J10, A8 |
| | TTC_{N} (N>4) | | | | | |
| C11 | 956212 | 15 | yphC | d | C/GTPase | J11, A7 |
| | CGT_{N}/GTC_{N} (N>3) | | | | | |
| C12 | 607260 | 13 | CPn0525 | | D/UFO | J9, A6, TR4, M7 |
| | ATGCT_{N}(N>2) | | | | | |
| C13 | 258158 | 15 | ypdP | d | D/UFO | J12, A11 |
| | ATTAA_{N} (N>2) | | | | | |
| C14 | 407929 | 15 | INT | d | C/sigma/rpsD C/flagelar secretion/flhA | J13 |
| | TTTCT_{N} (N>2) | | | | | |
| C15 | 396387 | 15 | CPn0352 | d | D/UFO | J14, A10 |

TABLE 2
Chlamydia pneumoniae strain CWL-029 TRs

| id | Position | length | period | genes | note | equivalent to |
|---|---|---|---|---|---|---|
| C1 | 7547 | 937 | 3 × 330 | INT | C/UFO/CPn0006 D/UFO/CPn0007 | A12 |
| C2 | 10807 | 178 | 2 × 89 | INT | D/UFO/CPn0007 D/UFO/CPn0008 | - (A12?) |
| C3 | 240764 | 45 | 2 × 13 | INT | D/oppA_4 D/oppB_1 | J2, A11 |
| C4 | 255447 | 35 | 2 × 14 | INT | C/UFO/CPn0214 | A9, J4 |
| C5 | 278045 | 26 | ⅔ × 8 | INT | C/CPn0240 C/CPn0241 | A8, J5 |
| C6 | 341108 | 32 | 2 × 15 | lpxD | D/UDP-acyltransferase | J6, A7 |
| C7 | 379100 | 40 | — | INT | many erased, C/CPn0333 D/CPn0334 | A6, J7 |
| C8 | 432020 | 330 | — | hctB | many erased, C/histone like | J8, A5, M1, TR1 |
| C9 | 451458 | 30 | 2 × 15 | CPn0405 | C/UFO | J9, A4 |
| C10 | 492298 | 20 | 4 × 6 | pmp_6 | D | J10, A3 |
| C11 | 568858 | 55 | 2 × 18 | CPn0487 | C/UFO | A2, J11 |
| C12 | 662224 | 80 | — | INT | many erased, C/murA D/UFOCPn0572 | J12 A1 M9 TR4 |
| C13 | 916873 | 70 | — | CPn0809 | many erased C/UFO | J13, A16, M11, |
| | | | | | | TR7 |
| C14 | 984289 | 30 | 2 × 13 | rodA | D/rod shape protein | J14, A15 |
| C15 | 1028449 | 26 | 2 × 13 | CPn0897 | C/phosphohydrolase | J15, A14 |
| C16 | 1085124 | 18 | 3 × 6 | glgA | C/glycogen synthase | J16, A13 |

TABLE 3
Chlamydia pneumoniae strain CWL-029 LRs (inverse)

| id | First | second | length | first | Second | equivalent to |
|---|---|---|---|---|---|---|
| C1 | 207095 | 208884 | 35 | D/CPnO165/UFO | C/CPnO169IUFO | J1 A1 |
| C2 | 493543 | 506266 | 23 | D/pmp_6 | INT | J3 A3 |
| C3 | 954974 | 955029 | 32 | C/CPn0843 | C/CPn0843 | J2 A2 |

TABLE 4
Chlamydia pneumoniae strain CWL-029 LRs (direct)

| id | First | second | length | first | Second | equivalent to |
|---|---|---|---|---|---|---|
| C1 | 26238 | 29415 | 23 | D/pmp_4.2 | D/pmp_5.2 | — |
| C2 | 234959 | 236693 | 27 | D/oppA_1 | D/oppA_2 | J1 A10 |
| C3 | 259232 | 259385 | 26 | INT | D/tgt/tRNA transferase | J2 A9 |
| C4 | 290023 | 292838 | 40 | C/CPn0255/UFO | INT | J3 A8 |
| C5 | 415142 | 416513 | 31 | D/CPn0369/UFO | D/CPn0370/UFO | J4 A6 |
| C6 | 495909 | 498766 | 23 | D/pmp_7 | D/pmp_8 | J6 A4 |
| C7 | 501979 | 514804 | 24 | D/pmp_9 | D/pmp_13 | J7 A3 |
| C8 | 522778 | 525176 | 28 | C/CPn0457/UFO | C/CPn0458/UFO | J8 A2 |
| C9 | 528528 | 530945 | 29 | C/CPn0461/UFO | C/CPn0462/UFO | J9 A1 |
| C10 | 1111630 | 1113279 | 1650 | D/glmS/amynotransferase | D/yccA_transport trunc | A13 |
| | | | | D/tyrP_1/transport | D/tyrP_2/transport | |

TABLE 5
Chlamydia pneumoniae strain AR 39 SSRs

| id | Position | length | gene | sense | Note | equivalent to |
|---|---|---|---|---|---|---|
| | G(C)_{N}(N>11) | | | | | |
| A1 | 334377 | 13 | CP0303 | d | D/PmpG | C6 |
| A2 | 782709 | 14 | CP0730 | d | C/UFO | C4 J4 |
| A3 | 820588 | 14 | CP0761 | d | C/PmpG | C3 J3 |
| A4 | 830377 | 14 | INT | d | C/CP0766/UFO C/CP0767/UFO | J1 C1 |
| A5 | 861807 | 15 | INT | | C/CP0795 C/CP0796 | C8 J7 |
| | ACG_{N}/GAC_{N} (N>3) | | | | | |
| A6 | 234314 | 13 | CP0228 | d | C/UFO | J9 C12 TR4 M7 |
| | GAA_{N} (N>4) | | | | | |
| A7 | 1115211 | 15 | CP1025 | d | D/GTP_binding | J11 C11 |
| | GGA_{N} (N>4) | | | | | |
| A8 | 920865 | 15 | CP0857 | d | C/FtsH | C10 J10 |
| | GGT_{N}/GTG_{N} (N>3) | | | | | |
| A9 | 213173 | 13 | INT | d | C/CP0209 C/CP0211 ABC transporters | C9 J8 |
| | AGAAA_{N}(N>2) | | | | | |
| A10 | 444793 | 15 | INT | d | C/CP0406/UFO C/CP0408/ATP carrier | J14 C15 |
| | AGCAT_{N} (N>2) | | | | | |
| A11 | 583107 | 15 | CP0548 | d | C/UFO | J12 C13 |
| | TTAAT_{N} (N>2) | | | | | |
| A12 | 433252 | 15 | CP0415 | d | D/reductoisomerase | — |

TABLE 6
Chlamydia pneumoniae strain AR 39 TRs

| id | Position | length | period | genes | Note | equivalent to |
|---|---|---|---|---|---|---|
| A1 | 179248 | 135 | — | INT | C/CP0177/UFO D/CP0178/transferase | J12 C12 M9 TR4 |
| A2 | 272675 | 43 | ⅔ × 13 | CP0267 | D/UFO | C11 J11 |
| A3 | 349273 | 18 | 3 × 6 | CP0309 | C/PmpG | J10 C10 |
| A4 | 389712 | 30 | 2 × 15 | CP0350 | D/UFO | C9 J9 |
| A5 | 408951 | 235 | — | CP0371 | D/Nucleoprotein | J8 C8 M1 TR1 |
| A6 | 462041 | 145 | — | INT | C/UFO/CP0424 D/UFO/CP0425 | C7 J7 |
| A7 | 500140 | 33 | 2 × 15 | CP0456 | C/UDP-transferase | C6 J6 |
| A8 | 563145 | 200 | — | INT | D/UFO/CP0521 D/UFO/CP0522 | C5 J5 |
| A9 | 585797 | 35 | 2 × 14 | CP0551 | D/UFO | C4 J4 |
| A10 | 587034 | 24 | ¾ × 7 | INT | D/UFO/CP0551 D/UFO/CP0553 | (J3?) |
| A11 | 600477 | 46 | 2 × 13 | INT | C/UFO/CP0568 C/UFO/CP0569 | C3 J2 |
| A12 | 832710 | 990 | 3 × 330 | CP0769 | C/UFO | C1 |
| A13 | 986268 | 18 | 3 × 6 | CP0911 | D/glycogen synthase | J16 C16 |
| A14 | 1042952 | 37 | 2 × 13 | CP0969 | D/UFO | J15 C15 |
| A15 | 1087106 | 43 | 2 × 13 | CP1002 | C/MrdB | J14 C14 |
| A16 | 1154483 | 40 | — | CP1062 | D/UFO | C13 J13 M11 TR7 |

TABLE 7
Chlamydia pneumoniae strain AR 39 LRs (inverse)

| id | First | second | length | First | second | equivalent to |
|---|---|---|---|---|---|---|
| A1 | 632368 | 634157 | 35 | D/CP0602/UFO | C/CP0606/UFO | J1 C1 |
| A2 | 1116377 | 1116432 | 32 | D/CP1026 | D/CP1026 frameshifted | C3 J2 |
| A3 | 335302 | 348025 | 23 | D/CP0303/pmpG | C/CP0309/pmpG | C2 J3 |

TABLE 8
Chlamydia pneumoniae strain AR 39 LRs (direct)

| id | first | second | length | First | second | equivalent to |
|---|---|---|---|---|---|---|
| A1 | 310615 | 313032 | 29 | D/CP0290/UFO | D/CP0291/UFO | C9 J9 |
| A2 | 316385 | 318783 | 28 | D/CP0294/UFO | D/CP0295/UFO | C8 J8 |
| A3 | 326762 | 339588 | 24 | C/CP0299/pmpG | C/CP0306/pmpG | J7 C7 |
| A4 | 342802 | 345659 | 23 | C/CP0307/pmpG | C/CP0308/pmpG | J6 C6 |
| A5 | 349362 | 349755 | 47 | C/CP0309/pmpG | C/CP0309/pmpG | J5 |
| A6 | 424652 | 426023 | 31 | C/CP0387/UFO | C/CP0388/UFO | J4 C5 |
| A7 | 540623 | 541657 | 449/365 | C/CP0493/UFO | C/CP0495/UFO | — |
| A8 | 548403 | 551218 | 40 | INT | D/CP0506/UFO | J3 C4 |
| A9 | 581869 | 582022 | 26 | C/CP0546/tRNA transferase | INT | J2 C3 |
| A10 | 604567 | 606301 | 27 | C/CP0571/ABC tr | C/CP0572/ABC tr | J1 C2 |
| A11 | 811759 | 814936 | 24 | INT | INT | — |
| A12 | 947563 | 948860 | 1144 | C/CP0878/UFO | C/CP0879/UFO | — |
| A13 | 956482 | 958131 | 1650 | C/CP0888/UFO | C/CP0891/permease | C10 |
| | | | | C/CP0889/permease | | |

TABLE 9
Chlamydia pneumoniae strain J138 SSRs

| id | position | length | gene | sense | note | equivalent to |
|---|---|---|---|---|---|---|
| | C(G)_{N}(N>11) | | | | | |
| J1 | 10806 | 14 | INT | d | D/UFO/CPj0007 D/UFO/CPj0008 | C1 A4 |
| J2 | 13350 | 13 | INT | d | D/UFO/CPj0009 D/UFO/CPj0010 | C2 |
| J3 | 20597 | 13 | pmp_2_1 | d | D | C3 A3 |
| J4 | 58475 | 14 | CPj0043 | d | D/UFO | C4 A2 |
| J5 | 506847 | 14 | pmp_10 | d | C | C6 A1 |
| J6 | 1205090 | 12 | CPj1054 | d | D/UFO | C7 |
| J7 | 1207641 | 16 | INT | d | D/UFO/CPj1054 D/UFO/CPj1055 grey hol | C8 A5 |
| | ACC_{N}/CAC_{N} (N>3) | | | | | |
| J8 | 628050 | 14 | CPj0542 | d | D/ABC transp | C9 A9 |
| | CGT_{N}/GTC_{N} (N>3) | | | | | |
| J9 | 606910 | 14 | CPj0525 | d | D/UFO | C12 A6, TR4, M7 |
| | TCC_{N} (N>4) | | | | | |
| J10 | 1148562 | 15 | ftsH | d | D | C10 A8 |
| | TTC_{N}/TTC_{N} (N>4) | | | | | |
| J11 | 955863 | 15 | yphC | d | C/GTPase | C11 A7 |
| | ATGCT_{N}(N>2) | | | | | |
| J12 | 258110 | 15 | ypdP | d | D/UFO | C13 A11 |
| | ATTAA_{N} (N>2) | | | | | |
| J13 | 407968 | 15 | flhA | d | C | C14 |
| | TTTCT_{N} (N>2) | | | | | |
| J14 | 396425 | 15 | CPj0352 | d | D | C15 A10 |

TABLE 10
Chlamydia pneumoniae strain J138 TRs

| id | position | length | period | genes | note | equivalent to |
|---|---|---|---|---|---|---|
| J1 | 127027 | 80 | 2 × 40 | htrB_1 | C/acyltransferase | — |
| J2 | 240709 | 38 | 2 × 13 | INT | D/oppA_4 D/oppB_1 | C3 A11 |
| J3 | 254172 | 28 | 4 × 7 | CPj0213 | C/UFO | - (A10?) |
| J4 | 255396 | 38 | 2 × 17 | CPj0214 | C/UFO | A9 C4 |
| J5 | 277997 | 60 | — | INT | C/UFO/CPj0240 C/UFO/CPj0241 | C5 A8 |
| J6 | 341060 | 30 | 2 × 15 | lpxD | D/UDP-acyltransferase | C6 A7 |
| J7 | 379052 | 140 | — | INT | C//ltuB/CPj0333 D/CPj0334 | A6 C7 |
| J8 | 432059 | 300 | — | hctB | C/histone like | C8 A5 M1 TR1 |
| J9 | 451497 | 30 | 2 × 15 | CPj0405 | C/UFO | C9 A4 |
| J10 | 491945 | 25 | ¾ × 6 | pmp_6 | D | C10 A3 |
| J11 | 568506 | 56 | 2 × 18 | CPj0487 | C/UFO | A2 C11 |
| J12 | 661875 | 120 | — | INT | C/murA D/CPj0572 | C12 A1 M9 TR4 |
| J13 | 916525 | 80 | — | CPj0809 | C/UFO | A16 C13 M11 TR9 |
| J14 | 983940 | 43 | 2 × 13 | rodA | D | C14 A15 |
| J15 | 1028100 | 37 | 2 × 13 | CPj0897 | C/phosphoydrolase | C15 A14 |
| J16 | 1084803 | 18 | 3 × 6 | glgA | C/glycogen synthase | C16 A13 |

TABLE 11
Chlamydia pneumoniae strain J138 LRs (inverse)

| id | First | second | length | first | second | equivalent to |
|---|---|---|---|---|---|---|
| J1 | 207048 | 208837 | 35 | D/CPj0165/UFO | C/CPj0169/UFO | C1 A1 |
| J2 | 954625 | 954680 | 32 | INT-C/CPj0843/UFO | C/CPj0843/UFO | C3 A2 |
| J3 | 493190 | 505913 | 23 | D/pmp_6 | C/pmp_10 | C2 A3 |

TABLE 12
Chlamydia pneumoniae strain J138 LRs (direct)

| id | first | second | length | first | second | equivalent to |
|---|---|---|---|---|---|---|
| J1 | 234904 | 236638 | 27 | D/oppA_1 | D/oppA_2 | C2 A10 |
| J2 | 259184 | 259337 | 26 | INT | D/tgt | C3 A9 |
| J3 | 289975 | 292790 | 40 | C/CPj0255/UFO | C/CPj0259/UFO | C4 A8 |
| J4 | 415181 | 416552 | 31 | D/CPj0369/UFO | D/CPj0370/UFO | C5 A6 |
| J5 | 491436 | 491829 | 47 | D/pmp_6 | D/pmp_6 | A5 |
| J6 | 495556 | 498413 | 23 | D/pmp_7 | D/pmp_8 | C6 A4 |
| J7 | 501626 | 514452 | 24 | D/pmp_9 | D/pmp_9 (2pmp9...) | C7 A3 |
| J8 | 522427 | 524825 | 28 | C/CPj0457/UFO | C/CPj0458/UFO | C8 A2 |
| J9 | 528177 | 530594 | 29 | C/CPj0461/UFO | C/CPj0462/UFO | C9 A1 |

TABLE 13
Chlamydia trachomatis strain D/UW-3/Cx SSRs

| id | position | length | gene | sense | note | equivalent to |
|---|---|---|---|---|---|---|
| | C(G)_{N}(N>11) | | | | | |
| TR1 | 291810 | 12 | INT | d | C/CT259 D/CT260 | — |
| | GT_{N} (N>5) | | | | | |
| TR2 | 964233 | 12 | ftsY | d | C/cell division | — |
| | ATT_{N} (N>4) | | | | | |
| TR3 | 1008839 | 15 | CT857 | d | D/UFO | — |
| | CGT_{N} (N>3) | | | | | |
| TR4 | 456967 | 15 | CT398 | d | D/UFO | M7, J9 A6, C12 |
| | GCA_{N} (N>4) | | | | | |
| TR5 | 531772 | 15 | CT456 | | D/UFO | — |
| | TGCAA_{N} (N>2) | | | | | |
| TR6 | 687502 | 15 | uvrD | d | D | — |

TABLE 14
Chlamydia trachomatis strain D/UW-3/Cx TRs

| id | position | Length | period | Genes | note | equivalent to |
|---|---|---|---|---|---|---|
| TR1 | 51545 | 400 | 15 bp - | hctB | many erased, D/histone like | M1 J8 A5 C8 |
| TR2 | 511072 | 53 | 2 × 15 | tsp | D/protease | — |
| TR3 | 527891 | 53 | 2 × 17 | argS | D/tRNA transferase | — |
| TR4 | 531363 | 450 | 3 × 150 | CT456 | D/UFO | J12 A1 C12 M9 |
| TR5 | 532487 | 18 | 3 × 6 | CT456 | D/UFO | — |
| TR6 | 613891 | 18 | 3 × 6 | dnaE | D/DNA pol III | — |
| TR7 | 650720 | 140 | — | CT578 | D/UFO | M11 J13 A16 C13 |
| TR8 | 657611 | 58 | 2 × 13 | gp6D | D/UFO/plasmid paralog | — |
| TR9 | 861061 | 40 | 2 × 16 | CT741 | C/UFO | — |
| TR10 | 984536 | 45 | 2 × 13 | INT | D/tRNASer_4 D/CT837 | — |

TABLE 15
Chlamydia trachomatis strain D/UW-3/Cx LRs (direct)

| id | first | second | Length | first | second |
|---|---|---|---|---|---|
| TR1 | 485249 | 574902 | 25 | tRNASer_3 | TRNASer_2 |
| TR2 | 853782 | 875828 | 5474 | rRNA + tRNA | rRNA + tRNA |

TABLE 16
Chlamydia muridarum SSRs

| id | position | length | gene | sense | Note | equivalent to |
|---|---|---|---|---|---|---|
| | C(G)_{N}(N>11) | | | | | |
| M1 | 501838 | 12 | TC0436 | d | C/phospholipase | — |
| M2 | 496505 | 12 | INT | i | C/TC0432 C/TC0433 phospholipases | — |
| M3 | 542159 | 13 | TC0447 | i | D/phospholipase | — |
| | AC_{N} (N>5) | | | | | |
| M4 | 787670 | 12 | TC0662 | d | D/UFO | — |
| | ACA_{N} (N>4) | | | | | |
| M5 | 1001176 | 15 | TC0868 | d | D/UFO | — |
| | AGC_{N} (N>4) | | | | | |
| M6 | 212303 | 17 | TC0181 | d | C | — |
| | CGT_{N} (N>3) | | | | | |
| M7 | 807276 | 15 | TC0677 | d | D/UFO | J9 C12 TR4 A6 |
| | CCTCC_{N} (N>2) | | | | | |
| M8 | 889812 | 15 | TC0750 | d | D/UFO | — |
| | GAGAG_{N} (N>2) | | | | | |
| M9 | 272616 | 15 | TC0235 | d | C/UFO | — |

TABLE 17
Chlamydia muridarum TRs

| id | position | length | period | genes | note | similar to |
|---|---|---|---|---|---|---|
| M1 | 369850 | 450 | — | TC0337 | C/UFO | TR1 J8 A5 C8 |
| M2 | 452977 | 50 | 4 × 14 | TC0392 | D/UFO | — |
| M3 | 602448 | 48 | 2 × 21 | TC0500 | C/UFO | — |
| M4 | 715922 | 975 | 5 × 201 | TC0602 | C/helicase | — |
| M5 | 738193 | 37 | 2 × 17 | TC0618 | C/dehydrogenase | — |
| M6 | 756226 | 55 | 2 × 13 | TC0634 | C/UFO | — |
| M7 | 758966 | 22 | 2 × 13 | TC0635 | C/UFO | — |
| M8 | 871834 | 44 | 2 × 13 | TC0733 | C/SecDF | — |
| M9 | 881447 | 930 | 3 × 330 | TC0741 | D/UFO | J12 A1 TR4 C12 |
| M10 | 985017 | 23 | 2 × 13 | INT | D/TC0850/type III secretion | — |
| | | | | | D/TC0853/type III membrane | |
| M11 | 1000266 | 80 | — | TC0867 | D/UFO | TR7 J13 C13 A16 |
| M12 | 1036721 | 18 | 3 × 6 | TC0898 | D/helicase/uvrD | — |

TABLE 18
Chlamydia muridarum LRs (inverse)

| | first | second | length | First | Second |
|---|---|---|---|---|---|
| M1 | 93051 | 985895 | 23 | C/TC0080/trigger factor | D/TC0853/type III |
| M2 | 495386 | 533316 | 1150 | C/TC0432/phospholipase | D/TC0440/phospholipase |
| M3 | 497071 | 533316 | 978 | C/TC0433/phospholipase | D/TC0440/phospholipase |

TABLE 19
LRs (direct)

| | first | second | length | First | Second |
|---|---|---|---|---|---|
| M1 | 133478 | 151897 | 1050 | D/TC0113/UFO-INT | INT-rRNA |
| M2 | 134545 | 156503 | 800 | C/TC0114/UFO-INT | C/TC0130/UFO-INT |
| M3 | 236729 | 238122 | 25 | D/TC0204/permease | D/TC0205/permease |
| M4 | 495294 | 496810 | 1244 | C/TC0432/phospholipase | C/TC0433/phospholipase |
| M5 | 503667 | 513542 | 23 | D/TC0437/adherence | D/TC0438/adherence |
| M6 | 539556 | 540091 | 119 | D/TC0444/UFO | C/TC0445/UFO |
| M7 | 834344 | 923737 | 1050 | C/tRNA-Ser-3 | C/tRNA-Ser-4 |
| | | | | D/TC0696/ABC transport | D/TC0784/helicase |

Some interesting features can be observed from these data. First, repeated sequences are more frequent in C. pneumoniae strains than in non-C. pneumoniae strains. This is true for SSRs, TRs, and direct LRs (t-student test, P<0.05 for SSR and TR and P<0. 1 for LDR) (Table 20).

TABLE 20

| | SSR | TR | LDR | LIR | Multiplets |
|---|---|---|---|---|---|
| Cpn CW | 15 | 16 | 10 | 3 | 1 |
| Cpn A | 12 | 16 | 13 | 3 | 0 |
| Cpn J | 14 | 16 | 9 | 3 | 1 |
| Ctr | 6 | 10 | 2 | 0 | 0 |
| Cmu | 9 | 12 | 7 | 3 | |

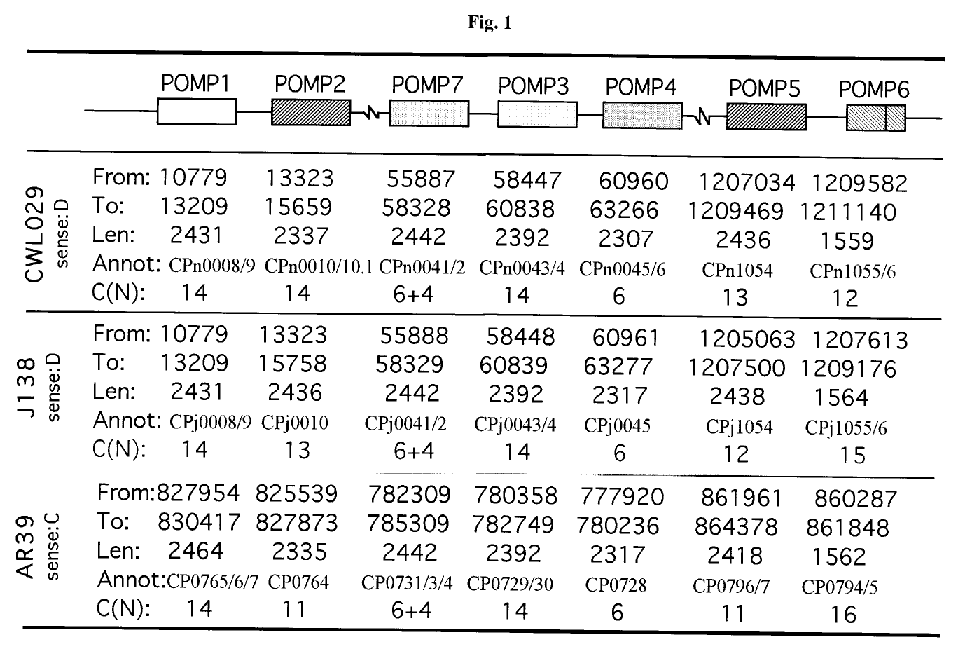

Second, several of these repeated sequences fall within the pmp locus. Indeed, even if larger numbers of LRs in C. pneumoniae can be attributed to the Pmp proteins, the larger numbers of SSRs and TRs are typically outside of these elements and possibly reflect other variation strategies. Third, this approach allowed us to discover a new family of seven genes encoding proteins that we call POMPs for polymorphic outer membrane proteins (FIG. 1).

Characterization of the POMPs

We performed a similarity search, motif analysis, and detection of transmembrane domains.

BLAST searches on the complete GenBank/EMBL/DDBJ database provided for no significant hits at E<10⁻¹⁰, except for C. pneumoniae sequences. The same result was observed when we performed a full search for orthologues in completely sequenced genomes (including C. trachomatis and C. muridarum). Finally, we carried out BLAST searches on the TIGR database of unfinished genomes, and against the fully sequenced, but still non-annotated genome of C. psitacci, also without positive results. Based on our searches, we concluded that POMP elements were specific to C. pneumoniae, perhaps having horizontally transferred after divergence with the other fully sequenced chlamydiae.

The analysis of the amino acid content revealed an excess of some residues, including cysteine, a residue that is characteristic of outer membrane proteins of C. pneumoniae (e.g. in Pmp; Melgosa et al, FEMS Lett., 112:199-204). We then determined whether the hydrophobicity profile, presence of putative transmembrane domains, and von Heijne's method for signal sequence recognition agreed in the prediction of a signal peptide. These methods indicated a signal peptide domain that would be cleaved at residue 51. We then used Klein's method for transmembrane region allocation, which predicted a transmembrane domain in residues 68-84. A similar result was obtained by using Top-pred. Using MTOP, we then predicted the membrane topology of the peptide. Results indicated that the N-terminal side should be inside, and the C-terminus outside. Thus, bioinformatic analyses consistently suggested that the POMP peptide was a membrane protein with one transmembrane segment, and a cytoplasmic N-terminus.

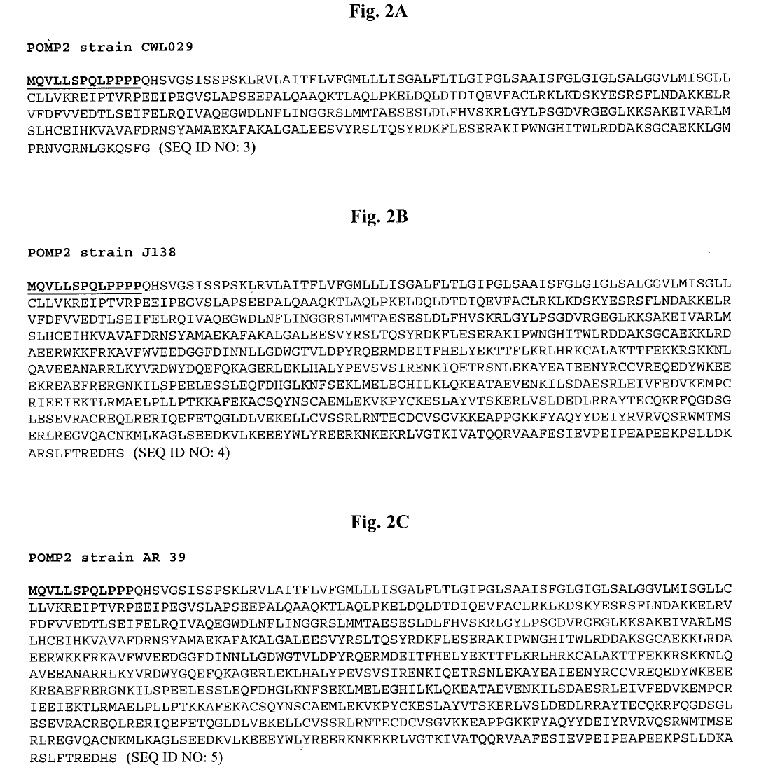

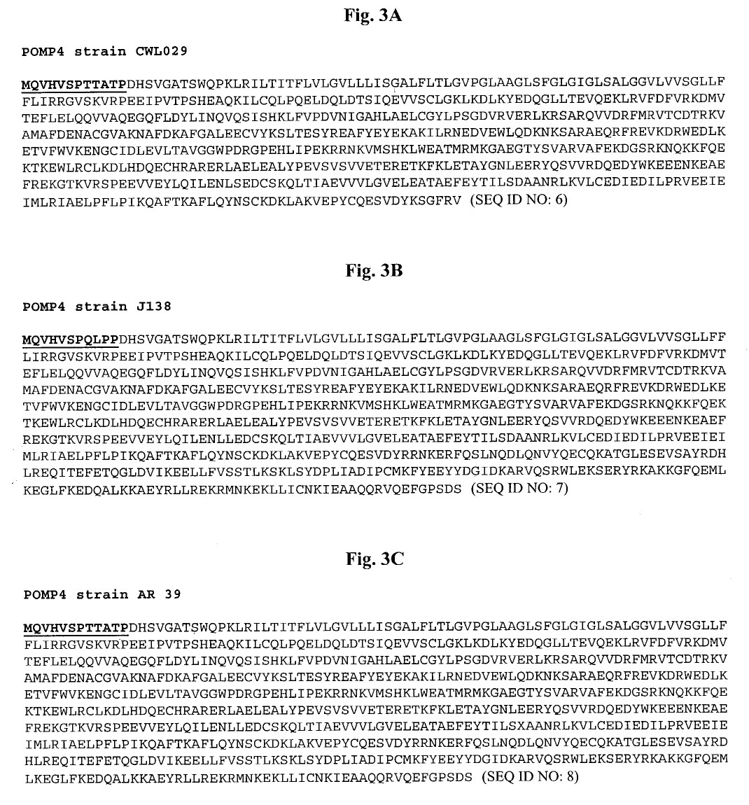

The putative amino acid sequences of POMP2 and POMP4 polypeptides are depicted in FIGS. 2A-2C and 3A-3C, respectively. The corresponding polynucleotide sequences are found at the region of the annotated sequence indicated in FIG. 1.

The identification of POMPs as a multigenic family restricted to C. pneumoniae strains implicates the POMP polynucleotides and polypeptides as being useful in the development of therapeutic and diagnostic agents, as described below.

Antibodies

The POMP polypeptides and polynucleotides of the invention (or variants thereof) or cells expressing the same can be used as immunogens to produce antibodies immunospecific for such polypeptides or polynucleotides respectively. Antibodies generated against POMP polypeptides or polynucleotides can be obtained by administering the polypeptides and/or polynucleotides, or epitope-bearing fragments of either or both, analogues of either or both, or cells expressing either or both, to an animal, preferably a nonhuman, using routine protocols. For preparation of monoclonal antibodies, any technique known in the art that provides antibodies produced by continuous cell line cultures can be used. Techniques for the production of single chain antibodies (U.S. Pat. No. 4,946,778) can be adapted to produce single chain antibodies to polypeptides or polynucleotides of this invention. Additionally, transgenic mice, or other organisms such as other mammals, may be used to express humanized antibodies immunospecific to the POMP polypeptides or polynucleotides of the invention. Phage display technology may be also utilized to select antibody genes with binding activities towards a POMP polypeptide of the invention, either from repertoires of PCR amplified v-genes of lymphocytes from humans screened for possessing anti-POMP, or from naive libraries. The affinity of these antibodies can also be improved by, for example, chain shuffling.

The above-described antibodies may be employed to isolate or to identify clones expressing a POMP polypeptide or polynucleotide of the invention to purify the polypeptide or polynucleotide by, for example, affinity chromatography. Antibodies against a POMP polypeptide or POMP polynucleotide may be employed to treat infections of C. pneumoniae.

In accordance with an aspect of the invention, there is provided the use of a POMP polynucleotide of the invention for therapeutic or prophylactic purposes, in particular genetic immunization. Among the particularly preferred embodiments of the invention are naturally occurring allelic variants of POMP polynucleotides and polypeptides encoded thereby. The use of a polynucleotide of the invention in genetic immunization will preferably employ a suitable delivery method such as direct injection of plasmid DNA into muscles, delivery of DNA complexed with specific protein carriers, coprecipitation of DNA with calcium phosphate, encapsulation of DNA in various forms of liposomes, particle bombardment, or in vivo infection using cloned retroviral vectors.

Drug Screening

POMP polypeptides and polynucleotides of the invention may also be used to assess the binding of small molecule substrates and ligands in, for example, cells, cell-free preparations, chemical libraries, and natural product mixtures. These substrates and ligands may be naturally occurring or may be structural or functional mimetics. In general, antagonists of POMP function may be employed for therapeutic and prophylactic purposes for treating infections of C. pneumoniae. The screening methods may simply measure the binding of a candidate compound to a POMP polypeptide or polynucleotide, or to cells or membranes bearing the polypeptide or polynucleotide, or a fusion protein of the polypeptide by means of a label directly or indirectly associated with the candidate compound. Alternatively, the screening method may involve competition with a labeled competitor. Further, these screening methods may test whether the candidate compound results in a signal generated by activation or inhibition of the POMP polypeptide, using detection systems appropriate to the cells expressing the POMP polypeptide. Inhibitors of activation are generally assayed in the presence of a known agonist and the effect on activation by the agonist by the presence of the candidate compound is observed.

POMP polypeptides may be used to identify membrane bound or soluble receptors, if any, for such polypeptide, through standard receptor binding techniques known in the art. These techniques include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (e.g., cells, cell membranes, cell supernatants, tissue extracts, bodily materials). Other methods include biophysical techniques such as surface plasmon resonance and spectroscopy. These screening methods may also be used to identify agonists and antagonists of the polypeptide that compete with the binding of the polypeptide to its receptor(s), if any. Standard methods for conducting such assays are well understood in the art.

Vaccines

The invention provides a method for inducing an immunological response in an individual, particularly a mammal, by inoculating the individual with a POMP polynucleotide and/or polypeptide, or a fragment or variant thereof, adequate to produce antibody and/or T cell immune response to protect that individual from an infection of C. pneumoniae.

A polypeptide of the invention may be used as an antigen for vaccination of a host to produce specific antibodies which protect against invasion of C. pneumoniae, for example by blocking adherence of bacteria to damaged tissue. Examples of tissue damage include wounds in skin or connective tissue caused, for example, by mechanical, chemical, thermal or radiation damage or by implantation of indwelling devices, or wounds in the mucous membranes, such as the mouth, throat, mammary glands, urethra, or vagina.

The invention also includes a vaccine formulation that includes an immunogenic recombinant polypeptide and/or polynucleotide of the invention together with a suitable carrier, such as a pharmaceutically acceptable carrier. Since the polypeptides and polynucleotides may be broken down in the stomach, each is preferably administered parenterally, including, for example, administration that is subcutaneous, intramuscular, intravenous, or intradermal. Formulations suitable for parenteral administration include aqueous and non-aqueous sterile injection solutions which may contain anti-oxidants, buffers, bacteriostatic compounds and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Diagnostics

Antibodies that specifically bind a POMP polypeptide may be used for the diagnosis of an infection of C. pneumoniae, or in assays to monitor patients being treated for an infection of C. pneumoniae. The antibodies useful for diagnostic purposes may be prepared in the same manner as those described above for therapeutics. Diagnostic assays for POMP polypeptides include methods that utilize the antibody and a label to detect POMP polypeptides in human body fluids or extracts of cells or tissues. The antibodies may be used with or without modification, and may be labeled by joining them, either covalently or non-covalently, with a reporter molecule. A wide variety of reporter molecules known in the art may be used. A variety of detection protocols (e.g., ELISA, RIA, and FACS) are also known in the art and provide a basis for diagnosing an infection of C. pneumoniae on the basis of detection of a POMP polypeptide.

POMP polynucleotides may also be used for diagnostic purposes. POMP polynucleotide sequences that may be used include antisense RNA and DNA molecules, and oligonucleotide sequences. The POMP polynucleotides may be used to detect and quantitate POMP expression in biopsied tissues. The diagnostic assay may be used to monitor an infection of C. pneumoniae during therapeutic intervention.

POMP polynucleotides may be used in Southern or northern analysis, dot blot, or other membrane-based technologies; in PCR technologies; or in dip stick, pIN, ELISA or chip assays utilizing fluids or tissues from patient biopsies to detect an infection of C. pneumoniae. Such qualitative or quantitative methods are well known in the art.

POMP polynucleotides may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable control sample, the labeled POMP polynucleotides have hybridized with polynucleotide sequences in the sample, indicating the presence of C. pneumoniae in the sample. Such assays may also be used to evaluate the efficacy of a particular therapeutic treatment regimen in animal studies, in clinical trials, or in monitoring the treatment of an individual patient.

Once disease is established and a treatment protocol is initiated, hybridization assays may be repeated on a regular basis to evaluate whether the expression in the patient is eliminated. The results obtained from successive assays may be used to show the efficacy of treatment over a period ranging from several days to months.

The foregoing results were obtained using the following methods.

Methods

An SSR is a strictly tandem repeat with n elements of a motif X (e.g., 3 CG in CGCGCG). Considering L, the length of the genome, the probability of not finding Xₙ anywhere is given by the formula:

P=(1−f_{X}ⁿ)^{L}

where f_{X} is the relative frequency of the motif X in the genome. We used a threshold p-value of 0.01 and searched for significant SSR elements with motifs ranging in length from 1 to 5 nucleotides, in all genomes of chlamydiae using standard pattern matching methods.

LR minimal length was defined through the use of a statistic of extremes that takes into account the composition in nucleotides and the length of the genome. For chlamydiae, this value is in the range of 25 nucleotides, which coincides with the minimal region of strict homology required for homologous recombination in E. coli and B. subtilis.

A small repeat was kept if it had at least the minimal significant length and if its two or more copies occur at short distances (<1 kbp). We searched for such repeats in sliding windows of 1000 bp, and for each window we computed the extreme statistics that allowed the definition of the length threshold in the window. These values varied slightly from window to window (in function of the window composition), but typically ranged from 12 to 14 bp. Then we inspected for more distinctive tandem repeats, by identifying repeats with occurrences at less than 50 bp apart, and for those with copies distant less than three times their length and by eye-checking all the others using dot-plots.

What is claimed is:

1. A method for determining the presence of a strain of chlamydia in a biological sample, said method comprising the steps of:
(a) providing a biological sample; and
(b) determining the presence of a polynucleotide containing a polymorphic repetitive sequence in a polynucleotide in said sample, said polymorphic repetitive sequence associated with a first strain of chlamydia and not associated with a second strain of chlamydia, wherein the presence of the polynucleotide containing said polymorphic repetitive sequence indicates the presence of said first strain of chlamydia.

2. The method of claim 1, wherein said chlamydia is C. pneuomoniae, C. trachomatis, C. psittaci, C. muridarum.

3. The method of claim 1, wherein said first strain is C. pneumoniae strain CWL-029, C. pneumoniae strain AR 39, C. pneumoniae strain J138, or C. trachomatis strain D/UW-3/Cx.

4. The method of claim 1, wherein said polymorphic repetitive sequence is a simple sequence repeat, a tandem repeat, or a large repeat.

5. The method of claim 1, wherein said sample is a biopsy sample, blood, serum, peripheral blood mononuclear cells, cerebrospinal fluid, urine, nasal secretion, or saliva.

6. The method of claim 1, wherein said determining of the presence of a polymorphic repetitive sequence comprises a polynucleotide detection step.

7. The method of claim 6, wherein said polynucleotide detection step comprises amplification of polynucleotide molecules that contain a polymorphic repetitive sequence.

8. A method for determining the presence of a plurality of strains of chlamydiae in a biological sample, said method comprising the steps of:
(a) providing a biological sample; and
(b) determining the presence in said biological sample of a plurality of polynucleotides, each containing a polymorphic repetitive sequence, wherein each polymorphic repetitive sequence is associated with one strain of chlamydia and not associated with another strain of chlamydiae, and wherein the presence of a polymorphic repetitive sequence indicates the presence of said strain of chlamydia associated with said polymorphic repetitive sequence, and absence of a polymorphic repetitive sequence indicates absence of said strain of chlamydia associated with said polymorphic repetitive sequence.

9. The method of claim 8, wherein said chlamydia is C. pneuomoniae, C. trachomatis, C. psittaci, C. muridarum.

10. The method of claim 8, wherein said strain is C. pneumoniae strain CWL-029, C. pneumoniae strain AR 39, C. pneumoniae strain J138, or C. trachomatis strain D/UW-3/Cx.

11. The method of claim 8, wherein said polymorphic repetitive sequence is a simple sequence repeat, a tandem repeat, or a large repeat.

12. The method of claim 8, wherein said sample is a biopsy sample, blood, serum, peripheral blood mononuclear cells, cerebrospinal fluid, urine, nasal secretion, or saliva.

13. The method of claim 8, wherein said determining of the presence of a polymorphic repetitive sequence comprises a polynucleotide detection step.

14. The method of claim 13, wherein said polynucleotide detection step comprises amplification of polynucleotide molecules that contain a polymorphic repetitive sequence.

15. A method for treating a chlamydial infection in a patient, said method comprising the steps of:
(a) providing a biological sample from the patient;
(b) determining the presence in said biological sample of a plurality of polynucleotides containing a polymorphic repetitive sequence, wherein each polymorphic repetitive sequence is associated with one strain of chlamydia and not associated with another strain of chlamydia, and wherein the presence of a polymorphic repetitive sequence indicates the presence of said strain of chlamydia associated with said polymorphic repetitive sequence, and absence of a polymorphic repetitive sequence indicates the absence of said strain of chlamydia associated with said polymorphic repetitive sequence; and
(c) administering to said patient anti-chlamydial agents that are effective against said strains of chlamydiae that are present in the biological sample.

16. The method of claim 15, wherein said chlamydia is C. pneuomoniae, C. trachomatis, C. psittaci, C. muridarum.

17. The method of claim 15, wherein said strain is C. pneumoniae strain CWL-029, C. pneumoniae strain AR 39, C. pneumoniae strain J138, or C. trachomatis strain D/UW-3/Cx.

18. The method of claim 15, wherein said polymorphic repetitive sequence is a simple sequence repeat, a tandem repeat, or a large repeat.

19. The method of claim 15, wherein said sample is a biopsy sample, blood, serum, peripheral blood mononuclear cells, cerebrospinal fluid, urine, nasal secretion, or saliva.

20. The method of claim 15, wherein said determining of the presence of a polymorphic repetitive sequence comprises a polynucleotide detection step.

21. The method of claim 20, wherein said polynucleotide detection step comprises amplification of polynucleotide molecules that contain a polymorphic repetitive sequence.

22. A purified polypeptide that is substantially identical to a POMP2 polypeptide selected from SEQ ID NO: 3, SEQ ID NO: 4, and SEQ ID NO: 5, or a POMP4 polypeptide selected from SEQ ID NO: 6, SEQ ID NO: 7, and SEQ ID NO: 8.

23. A purified polynucleotide encoding a polypeptide that is substantially identical to a POMP2 polypeptide selected from SEQ ID NO: 3, SEQ ID NO: 4, and SEQ ID NO: 5, or a POMP4 polypeptide selected from SEQ ID NO: 6, SEQ ID NO: 7, and SEQ ID NO: 8.

24. A method of immunizing a subject against an infection of C. pneumoniae, said method comprising administering to said subject a purified POMP polypeptide or an immunogenic fragment thereof in an amount sufficient to induce an immune response to said POMP polypeptide or fragment thereof, wherein said immune response immunizes the subject against an infection of C. pneumoniae.

25. An isolated antibody that specifically binds a POMP polypeptide of claim 22 or a fragment thereof.

26. A method of producing an immune response in an animal, said method comprising immunizing the animal with an effective amount of a POMP polypeptide, or immunogenic fragment thereof.

27. The method of claim 26, wherein said POMP polypeptide is a POMP2 or POMP4 polypeptide.


SEQUENCE LISTING (8 sequences)

SEQ ID NO: 1 - protein, 4 aa, Artificial Sequence
  VARIANT 4: Xaa = L, V or I
GGAX

SEQ ID NO: 2 - protein, 4 aa, Artificial Sequence
  VARIANT 2, 3: Xaa = any amino acid
FXXN

SEQ ID NO: 3 - protein, 334 aa, Chlamydia pneumoniae
MQVLLSPQLPPPPQHSVGSISSPSKLRVLAITFLVFGMLLLISGALFLTLGIPGLSAAISFGLGIGLSALGGVLMISGLLCLLVKREIPTVRPEEIPEGVSLAPSEEPALQAAQKTLAQLPKELDQLDTDIQEVFACLRKLKDSKYESRSFLNDAKKELRVFDFVVEDTLSEIFELRQIVAQEGWDLNFLINGGRSLMMTAESESLDLFHVSKRLGYLPSGDVRGEGLKKSAKEIVARLMSLHCEIHKVAVAFDRNSYAMAEKAFAKALGALEESVYRSLTQSYRDKFLESERAKIPWNGHITWLRDDAKSGCAEKKLGMPRNVGRNLGKQSFG

SEQ ID NO: 4 - protein, 811 aa, Chlamydia pneumoniae
MQVLLSPQLPPPPQHSVGSISSPSKLRVLAITFLVFGMLLLISGALFLTLGIPGLSAAISFGLGIGLSALGGVLMISGLLCLLVKREIPTVRPEEIPEGVSLAPSEEPALQAAQKTLAQLPKELDQLDTDIQEVFACLRKLKDSKYESRSFLNDAKKELRVFDFVVEDTLSEIFELRQIVAQEGWDLNFLINGGRSLMMTAESESLDLFHVSKRLGYLPSGDVRGEGLKKSAKEIVARLMSLHCEIHKVAVAFDRNSYAMAEKAFAKALGALEESVYRSLTQSYRDKFLESERAKIPWNGHITWLRDDAKSGCAEKKLRDAEERWKKFRKAVFWVEEDGGFDINNLLGDWGTVLDPYRQERMDEITFHELYEKTTFLKRLHRKCALAKTTFEKKRSKKNLQAVEEANARRLKYVRDWYDQEFQKAGERLEKLHALYPEVSVSIRENKIQETRSNLEKAYEAIEENYRCCVREQEDYWKEEEKREAEFRERGNKILSPEELESSLEQFDHGLKNFSEKLMELEGHILKLQKEATAEVENKILSDAESRLEIVFEDVKEMPCRIEEIEKTLRMAELPLLPTKKAFEKACSQYNSCAEMLEKVKPYCKESLAYVTSKERLVSLDEDLRRAYTECQKRFQGDSGLESEVRACREQLRERIQEFETQGLDLVEKELLCVSSRLRNTECDCVSGVKKEAPPGKKFYAQYYDEIYRVRVQSRWMTMSERLREGVQACNKMLKAGLSEEDKVLKEEEYWLYREERKNKEKRLVGTKIVATQQRVAAFESIEVPEIPEAPEEKPSLLDKARSLFTREDHS

SEQ ID NO: 5 - protein, 810 aa, Chlamydia pneumoniae
MQVLLSPQLPPPQHSVGSISSPSKLRVLAITFLVFGMLLLISGALFLTLGIPGLSAAISFGLGIGLSALGGVLMISGLLCLLVKREIPTVRPEEIPEGVSLAPSEEPALQAAQKTLAQLPKELDQLDTDIQEVFACLRKLKDSKYESRSFLNDAKKELRVFDFVVEDTLSEIFELRQIVAQEGWDLNFLINGGRSLMMTAESESLDLFHVSKRLGYLPSGDVRGEGLKKSAKEIVARLMSLHCEIHKVAVAFDRNSYAMAEKAFAKALGALEESVYRSLTQSYRDKFLESERAKIPWNGHITWLRDDAKSGCAEKKLRDAEERWKKFRKAVFWVEEDGGFDINNLLGDWGTVLDPYRQERMDEITFHELYEKTTFLKRLHRKCALAKTTFEKKRSKKNLQAVEEANARRLKYVRDWYGQEFQKAGERLEKLHALYPEVSVSIRENKIQETRSNLEKAYEAIEENYRCCVREQEDYWKEEEKREAEFRERGNKILSPEELESSLEQFDHGLKNFSEKLMELEGHILKLQKEATAEVENKILSDAESRLEIVFEDVKEMPCRIEEIEKTLRMAELPLLPTKKAFEKACSQYNSCAEMLEKVKPYCKESLAYVTSKERLVSLDEDLRRAYTECQKRFQGDSGLESEVRACREQLRERIQEFETQGLDLVEKELLCVSSRLRNTECDCVSGVKKEAPPGKKFYAQYYDEIYRVRVQSRWMTMSERLREGVQACNKMLKAGLSEEDKVLKEEEYWLYREERKNKEKRLVGTKIVATQQRVAAFESIEVPEIPEAPEEKPSLLDKARSLFTREDHS

SEQ ID NO: 6 - protein, 610 aa, Chlamydia pneumoniae
MQVHVSPTTATPDHSVGATSWQPKLRILTITFLVLGVLLLISGALFLTLGVPGLAAGLSFGLGIGLSALGGVLVVSGLLFFLIRRGVSKVRPEEIPVTPSHEAQKILCQLPQELDQLDTSIQEVVSCLGKLKDLKYEDQGLLTEVQEKLRVFDFVRKDMVTEFLELQQVVAQEGQFLDYLINQVQSISHKLFVPDVNIGAHLAELCGYLPSGDVRVERLKRSARQVVDRFMRVTCDTRKVAMAFDENACGVAKNAFDKAFGALEECVYKSLTESYREAFYEYEKAKILRNEDVEWLQDKNKSARAEQRFREVKDRWEDLKETVFWVKENGCIDLEVLTAVGGWPDRGPEHLIPEKRRNKVMSHKLWEATMRMKGAEGTYSVARVAFEKDGSRKNQKKFQEKTKEWLRCLKDLHDQECHRARERLAELEALYPEVSVSVVETERETKFKLETAYGNLEERYQSVVRDQEDYWKEEENKEAEFREKGTKVRSPEEVVEYLQILENLSEDCSKQLTIAEVVVLGVELEATAEFEYTILSDAANRLKVLCEDIEDILPRVEEIEIMLRIAELPFLPIKQAFTKAFLQYNSCKDKLAKVEPYCQESVDYKSGFRV

SEQ ID NO: 7 - protein, 770 aa, Chlamydia pneumoniae
MQVHVSPQLPPDHSVGATSWQPKLRILTITFLVLGVLLLISGALFLTLGVPGLAAGLSFGLGIGLSALGGVLVVSGLLFFLIRRGVSKVRPEEIPVTPSHEAQKILCQLPQELDQLDTSIQEVVSCLGKLKDLKYEDQGLLTEVQEKLRVFDFVRKDMVTEFLELQQVVAQEGQFLDYLINQVQSISHKLFVPDVNIGAHLAELCGYLPSGDVRVERLKRSARQVVDRFMRVTCDTRKVAMAFDENACGVAKNAFDKAFGALEECVYKSLTESYREAFYEYEKAKILRNEDVEWLQDKNKSARAEQRFREVKDRWEDLKETVFWVKENGCIDLEVLTAVGGWPDRGPEHLIPEKRRNKVMSHKLWEATMRMKGAEGTYSVARVAFEKDGSRKNQKKFQEKTKEWLRCLKDLHDQECHRARERLAELEALYPEVSVSVVETERETKFKLETAYGNLEERYQSVVRDQEDYWKEEENKEAEFREKGTKVRSPEEVVEYLQILENLLEDCSKQLTIAEVVVLGVELEATAEFEYTILSDAANRLKVLCEDIEDILPRVEEIEIMLRIAELPFLPIKQAFTKAFLQYNSCKDKLAKVEPYCQESVDYRRNKERFQSLNQDLQNVYQECQKATGLESEVSAYRDHLREQITEFETQGLDVIKEELLFVSSTLKSKLSYDPLIADIPCMKFYEEYYDGIDKARVQSRWLEKSERYRKAKKGFQEMLKEGLFKEDQALKKAEYRLLREKRMNKEKLLICNKIEAAQQRVQEFGPSDS

SEQ ID NO: 8 - protein, 771 aa, Chlamydia pneumoniae
  VARIANT 537: Xaa = Any Amino Acid
MQVHVSPTTATPDHSVGATSWQPKLRILTITFLVLGVLLLISGALFLTLGVPGLAAGLSFGLGIGLSALGGVLVVSGLLFFLIRRGVSKVRPEEIPVTPSHEAQKILCQLPQELDQLDTSIQEVVSCLGKLKDLKYEDQGLLTEVQEKLRVFDFVRKDMVTEFLELQQVVAQEGQFLDYLINQVQSISHKLFVPDVNIGAHLAELCGYLPSGDVRVERLKRSARQVVDRFMRVTCDTRKVAMAFDENACGVAKNAFDKAFGALEECVYKSLTESYREAFYEYEKAKILRNEDVEWLQDKNKSARAEQRFREVKDRWEDLKETVFWVKENGCIDLEVLTAVGGWPDRGPEHLIPEKRRNKVMSHKLWEATMRMKGAEGTYSVARVAFEKDGSRKNQKKFQEKTKEWLRCLKDLHDQECHRARERLAELEALYPEVSVSVVETERETKFKLETAYGNLEERYQSVVRDQEDYWKEEENKEAEFREKGTKVRSPEEVVEYLQILENLLEDCSKQLTIAEVVVLGVELEATAEFEYTILSXAANRLKVLCEDIEDILPRVEEIEIMLRIAELPFLPIKQAFTKAFLQYNSCKDKLAKVEPYCQESVDYRRNKERFQSLNQDLQNVYQECQKATGLESEVSAYRDHLREQITEFETQGLDVIKEELLFVSSTLKSKLSYDPLIADIPCMKFYEEYYDGIDKARVQSRWLEKSERYRKAKKGFQEMLKEGLFKEDQALKKAEYRLLREKRMNKEKLLICNKIEAAQQRVQEFGPSDS